(19) United States
(12) Patent Application Publication
(10) Pub. No.: US 2011/0165619 A1
(43) Pub. Date: Jul. 7, 2011

(54) Secretion Optimized Microorganism

(76) Inventors: JOHANNES BONGAERTS, DORMAGEN (DE); STEFAN EVERS, METTMANN (DE); KERSTIN FOH, WEINHEIM (DE); KARL-HEINZ MAURER, ERKRATH (DE)

(21) Appl. No.: 12/955,353

(22) Filed: Nov. 29, 2010

Related U.S. Application Data
(63) Continuation of application No. PCT/EP2009/056143, filed on May 20, 2009.

(30) Foreign Application Priority Data
May 29, 2008 (DE) 102008025791.5

(51) Int. Cl. C12P 21/02; C12N 1/00; C12N 1/21; C12N 9/02; C12N 9/08; C12N 9/52; C12N 9/28; C12N 9/42; C12N 9/16

(52) U.S. Cl. 435/69.1; 435/243; 435/252.35; 435/189; 435/192; 435/220; 435/202; 435/209; 435/196

(57) ABSTRACT

Proteins having a cofactor can be secreted in an improved manner in a microorganism belonging to the genus Streptomyces provided that the microorganism contains a nucleic acid sequence which is not naturally present in it and which comprises at least the following sequence sections: a) nucleic acid sequence coding for a protein containing a cofactor, and b) a nucleic acid sequence which is at least 20% identical to the sequence given in SEQ ID NO. 1, or at least 20% identical to the sequence given in SEQ ID NO. 3, or a nucleic acid sequence which is structurally homologous to at least one of these sequences, wherein the amino acid sequence encoded by nucleic acid sequence b) functionally cooperates with the amino acid sequence encoded by nucleic acid sequence a) so that at least the amino acid sequence encoded by nucleic acid sequence a) is secreted by the microorganism.

### CROSS REFERENCE TO RELATED APPLICATIONS

The present application is a continuation of International Patent Application No. PCT/EP2009/056143 filed 20 May 2009, which claims priority to German Patent Application No. 10 2008 025 791.5 filed 29 May 2008, both of which are incorporated herein by reference.

The invention is directed towards microorganisms containing a nucleic acid sequence that is not naturally present in them, and that includes at least the following sequence segments:
a) nucleic acid sequence coding for a protein having a cofactor, and
b) nucleic acid sequence that is at least 20% identical to the sequence stated in SEQ ID NO.1 or is at least 20% identical to the sequence stated in SEQ ID NO.3 or is a structurally homologous nucleic acid sequence to at least one of these sequences,
   wherein the amino acid sequence coded by nucleic acid sequence b) functionally interacts with the amino acid sequence coded by nucleic acid sequence a) in such a way that at least the amino acid sequence coded by nucleic acid sequence a) is secreted from the microorganism, with the proviso that the microorganism belongs to the genus Streptomyces. Microorganisms of this type can be used for improving biotechnological production processes for proteins comprising a cofactor. Consequently, the invention is further directed towards uses of microorganisms of this type, as well as processes in which such microorganisms are cultivated, particularly fermentative uses and processes.

The present invention is in the field of biotechnology, particularly the manufacture of valuable substances by fermentation of microorganisms capable of forming such valuable substances of interest. These include, for example, the manufacture of low molecular weight compounds (e.g., food supplements or pharmaceutically relevant compounds) or proteins, which, due to their diversity, there is a large range of industrial applications.

There exists substantial prior art covering fermentation of microorganisms, particularly on the industrial scale. It ranges from optimization of the strains in question with respect to rates of formation and nutrient utilization, through technical design of the fermentor, to recovery of valuable materials from the cells in question and/or fermentation medium. Both genetic and microbiological as well as process engineering and biochemical approaches are involved.

For economical production of proteins (e.g., enzymes), one generally seeks firstly to obtain the highest possible product yield in the fermentation, and secondly to eject the product from the producing organism by secretion from the cell into the production medium. This avoids costly digestion of the cells and, because less unwanted cell components have to be separated, significantly simplifies further purification and downstream processing. The majority of industrial enzymes are secreted naturally, particularly proteases and amylases which are employed in washing and cleaning agents. The genes of these enzymes have a signal sequence, often called the Sec-signal sequence, before the sequence that codes for the enzyme (or proenzyme in the case of proteases). This Sec-signal sequence codes an N-terminal signal peptide responsible for translocation of the unfolded enzyme over the cytoplasm membrane (see dependent secretion).

Moreover, Tat—(“Twin-arginine translocation”) dependent secretion of proteins is known from the prior art (see inter alia, Schaerlaekens et al., J. Biotechnol., Vol. 112, pp. 279-288 (2004)). This is conveyed over Tat-signal peptides. Various Tat-signal peptides from various species are known from the prior art, including E. coli and Bacillus subtilis, as well as from members of the genera Streptomyces and Corynebacterium.

International Patent Application Publication No. WO 2002/022667 shows that completely folded polypeptide chains are ejected over the Tat-secretion path. This secretion path is also suitable for secretion of proteins comprising a cofactor. It is therefore proposed to use the Tat-secretion path for heterologous expression of proteins. However, this application likewise shows that not every Tat-signal peptide in all microorganisms or in all bacteria also effects a corresponding secretion. For example, the PhoD-signal peptide from Bacillus subtilis is not detected from the Tat-secretion system of E. coli per se (see, Example 4 of WO 2002/022667), but rather only after genetic modification thereof (here by recombinant expression of two components of the B. subtilis Tat-secretion system). The article by Pop et al., J. of Biological Chemistry, Vol. 277(5), pp. 3268-3273 (2002) also comes to the same conclusion.

Therefore, a heterologous expression system that allows Tat mediated secretion of a cofactor-containing protein, particularly an enzyme, in different microorganisms cannot be determined from the prior art. In particular, this is not disclosed for bacteria of the genus Streptomyces. Furthermore, no such system is known for Streptomyces which enables a satisfactory product yield in fermentation.

Accordingly, the present invention seeks to improve biotechnological production of proteins, particularly those having a cofactor, especially by using bacteria of the genus Streptomyces. Additionally, the invention seeks to increase, in a fermentation process, the product yield of proteins, particularly those having a cofactor, again by using bacteria of the genus Streptomyces. In particular, a microorganism should be made available, especially one of the genus Streptomyces which secretes in an improved manner proteins having a cofactor, and whose use further preferably increases the product yield in a fermentation process.

Consequently, the present invention provides for a microorganism having a nucleic acid sequence that is not naturally present in it, and that includes at least the following sequence segments:
a) nucleic acid sequence coding for a protein having a cofactor, and
b) nucleic acid sequence that is at least 20% identical to the sequence stated in SEQ ID NO.1 or is at least 20% identical to the sequence stated in SEQ ID NO.3 or is a structurally homologous nucleic acid sequence to at least one of these sequences,
   wherein the amino acid sequence coded by nucleic acid sequence b) functionally interacts with the amino acid sequence coded by nucleic acid sequence a) in such a way that at least the amino acid sequence coded by nucleic acid sequence a) is secreted from the microorganism, with the proviso that the microorganism belongs to the genus Streptomyces.

It was surprisingly found that such nucleic acid sequences in bacteria of the genus Streptomyces effect secretion of proteins having a cofactor, especially from protein coded from a nucleic acid sequence a) normally localized in the cytosol of the cell and was therefore not secreted. Moreover, they affect this in such a degree that a microorganism of this type is suitable for biotechnological production of the cofactor-containing protein, especially in fermentation processes.

### BRIEF DESCRIPTION OF THE DRAWINGS

FIG. 1 illustrates construction of the expression plasmid pKF1 for the cytosolic expression of the heterologous choline oxidase in Streptomyces lividans. The shuttle vector pWHM3 is illustrated, in which the fusion PCR product from PermE*-promoter and choline oxidase gene, cod, was cloned over the HindIII and the EcoRI segment.

FIG. 2 illustrates construction of the expression vectors pVR19 and pVR22 for the secretory production of the heterologous choline oxidase in Streptomyces lividans. The expression vector pKF1 (see FIG. 1) is illustrated, into which the DNA sequence of the signal peptide of the gene SCO0624 or of the gene SCO6272 was introduced through insertion mutagenesis; SP=signal peptide (SP-SCO0624→pVR19, SP-SCO6272→pVR22).

FIG. 3 illustrates a qualitative activity test for hydrogen peroxide-forming enzymes on agar plates by means of 4-chloronaphthol. The Streptomyces lividans TK23 strains were compared with the empty vector pWHM3, with the vector for the cytoplasmatic expression, pKF1, and with the vectors for the Tat-dependent secretory production of the choline oxidase, pVR19 and pVR22. 50 μL, of each culture supernatant (samples taken after 48 h, 72 h and 96 h) were placed in stamped holes and incubated at room temperature for 1.5 h. The blue coloration showed the activity of the choline oxidase.

A microorganism belonging to the genus Streptomyces is understood to mean bacteria of the genus Streptomyces, which, according to the actual definition, is the sole genus within the family of Streptomycetaceae. In addition to the original genus Streptomyces, it also includes the earlier different genera Actinopycnidium, Actinosporangium, Chainia, Elytrosporangium, Kitasatoa, Microellobosporia and Streptoverticillium.

In the context of the present patent application, microorganisms of the genera Kitasatosporia, Kineosporia and Sporichthya are also considered to additionally belong to the genus Streptomyces. An overview of Streptomyces taxonomy is given in the publication Anderson et al., “The taxonomy of Streptomyces and related genera”, Intl. J. Syst. Evol. Microbiol., Vol. 51, pp. 797-814 (2001), to which explicit reference is made and whose disclosure is incorporated in its entirety into the contents of the present patent application.

Microorganisms belonging to the genus Streptomyces are in particular gram-positive, aerobic representatives of Actinomycetes having a DNA G-C content of especially 69-78 mol % and which mostly form an extensive, branched substrate mycelium and air mycelium. A characteristic of microorganisms of the genus Streptomyces is the occurrence of the LL-isomer of diamino pimelic acid as a component of the cell wall.

In this context, “not naturally present” means that the nucleic acid sequence is not an innate sequence of the microorganism (i.e., is not present in this form in the wild type form of the microorganism or cannot be isolated from it). Consequently, a natural nucleic acid sequence would therefore be present in the genome of the given microorganism per se (i.e., in its wild type form). In contrast, a sequence of this type would be introduced into microorganisms according to the invention, preferably introduced in a targeted manner, or produced in them, for example, preferably with the aid of genetic engineering processes. Therefore this sequence is not naturally present in the particular microorganism, so that the microorganism is enriched by this sequence. This sequence is preferably expressed by the microorganism. Accordingly, the nucleic acid sequence in a microorganism according to the invention preferably further contains, in addition to nucleic acid sequences a) and b) described below, at least one or more sequences, especially promoter sequences for expressing nucleic acid sequences a) and b).

Accordingly, the nucleic acid sequence in a microorganism according to the invention contains at least two sequence segments, namely nucleic acid sequences a) and b), and preferably further contains one or more sequences, particularly promoter sequences, for expressing nucleic acid sequences a) and b). Nucleic acid sequence a) codes here for a protein having a cofactor (i.e., the protein that is secreted from the microorganism and thereby intended to be ejected from it). Nucleic acid sequence b) codes here for an amino acid sequence that interacts with a translocation system used from the microorganism; thus, in the present case from a bacterium of the genus Streptomyces so that at least the amino acid sequence coded by nucleic acid sequence a) is secreted from the microorganism. Consequently, the amino acid sequence coded from this nucleic acid sequence b) binds directly or indirectly to at least one component of the translocation system of the microorganism according to the invention. Direct binding is understood to mean a direct interaction that can be covalent or non-covalent; indirect binding is understood to mean that the interaction can occur over one or more additional components, especially proteins or other molecules that act as an adapter and accordingly have a bridging function between the amino acid sequence coded by nucleic acid sequence b) and a component of the bacterial translocation system, wherein here as well the interactions can be covalent or non covalent.

The translocation system used preferably concerns a Tat-dependent secretion (i.e., uses at least one component of the Tat-secretion system). Nucleic acid sequence b) therefore codes for a Tat-signal sequence (Tat-signal peptide) that is functional in Streptomyces and enables secretion of the amino acid sequence coded by nucleic acid sequence a). In this way, due to the presence of the amino acid sequence coded by nucleic acid sequence b), a cofactor-containing protein (coded by nucleic acid sequence a)) is secreted from bacteria of the genus Streptomyces.

Amino acid sequences coded by nucleic acid sequences b) and a) can be components of the same polypeptide chain, but can also be present on polypeptide chains that are not covalently bound with one another. It is possible, for example, that non-covalently bound polypeptide chains nevertheless interact with one another in such a way that the cofactor-containing protein coded by nucleic acid sequence a) is also ejected from the cell due to the existence of the amino acid sequence coded by nucleic acid sequence b). By functional coupling/functional interaction of the amino acid sequence coded by nucleic acid sequence b) and that of the cofactor-containing protein coded by nucleic acid sequence a) as described, the issue therefore is to understand that the cofactor-containing protein coded by nucleic acid sequence a) is ejected out of the cell due to the existence of the amino acid sequence coded by nucleic acid sequence b). Without the presence of the amino acid sequence coded by nucleic acid sequence b) in the cell, secretion of the cofactor-containing protein coded by nucleic acid sequence a) would be diminished or not present at all. An exemplary and particularly preferred functional interaction of this type is achieved in that the amino acid sequence coded by nucleic acid sequence b) and the amino acid sequence coded by nucleic acid sequence a) are components of the same polypeptide chain, at least inside the cell. In principle, however, the amino acid sequences coded from the relevant nucleic acid sequences a) and b) can also be present on separate polypeptide chains as long as the functional interaction of both sequences—i.e., the advantage and/or necessity of the presence of the amino acid sequence coded by nucleic acid sequence b) for the secretion of the cofactor-containing protein coded by nucleic acid sequence a)—is given, at least inside the cell, for example, by direct or indirect binding of both amino acid sequences to one another, wherein all bonds can be covalent or non-covalent.

In comparative experiments a functional interaction of this type is determined wherein a first microorganism containing a nucleic acid sequence according to the invention having at least one nucleic acid sequence b) and one nucleic acid sequence a) and expresses them, is compared with a second microorganism that differs from the first microorganism only in that it does not contain nucleic acid sequence b). Both microorganisms were cultivated under the same conditions, wherein the conditions were chosen such that at least the first microorganism expresses and secretes the cofactor-containing protein coded by nucleic acid sequence a). The presence of a functional interaction is demonstrated by increased secretion of the cofactor-containing protein coded by nucleic acid sequence a) by the first microorganism when compared with the second microorganism.

Nucleic acid sequence b) in this regard is at least 20% identical to the nucleic acid sequence listed in SEQ ID NO.1 or at least 20% identical to the amino acid sequence coded by it (listed in SEQ ID NO. 2) or at least 20% identical to the nucleic acid sequence in SEQ ID NO.3 or at least 20% identical to the amino acid sequence coded by it (listed in SEQ ID NO.4), each based on total length of the listed sequences. Nucleic acid sequence b) is increasingly preferably identical to at least 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% and most preferably 100% identical to the nucleic acid sequence listed in SEQ ID NO. 1, or to at least 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% and most preferably 100% identical to the amino acid sequence coded by it (listed in SEQ ID NO:2), or to at least 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% and quite particularly preferably 100% identical to the nucleic acid sequence listed in SEQ ID NO.3, or to at least 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% and quite particularly preferably 100% identical to the amino acid sequence coded by it (listed in SEQ ID NO.4). Unexpectedly, these sequences enable an efficient Tat-dependent secretion of a cofactor-containing protein in bacteria of the genus Streptomyces.

Instead of the cited sequences that enable secretion of a cofactor-containing protein, their structurally homologous sequences can also be used. A structurally homologous nucleic acid sequence is understood to mean a sequence that codes an amino acid sequence whose order of amino acids causes such a spatial folding of this sequence that it interacts with the employed translocation system of Streptomyces so that the cofactor-containing protein of the translocation system is ejected from the Streptomyces cell. Consequently, the amino acid sequence coded by this nucleic acid sequence binds directly or indirectly to at least one component of the translocation system of the microorganism according to the invention. A direct binding is understood to mean a direct interaction; an indirect binding is understood to mean that the interaction can occur over one or more additional components, especially proteins or other molecules that act as an adapter and accordingly have a bridging function between the amino acid sequence coded by the structurally homologous nucleic acid sequence and a component of the bacterial translocation system.

A preferred structurally homologous nucleic acid sequence according to the invention codes for a Tat signal peptide containing three motifs: a positively charged N-terminal motif, a hydrophobic region and a C-terminal region that comprises a short consensus motif (A-x-A) and preferably ends with this motif that specifies the cleavage site by a signal peptidase. A Tat signal peptide coded by a structurally homologous nucleic acid sequence according to the invention likewise preferably includes a consensus sequence [ST]-R-R-X-F-L-K. The amino acids are listed using the one letter code commonly used by experts for amino acids in protein sequences, wherein x is any amino acid in the protein sequence and ST is serine or threonine. It is important that the amino acid sequence coded by the structurally homologous nucleic acid sequence is not just any Tat signal peptide of the prior art, but rather is an amino acid sequence recognized by the translocation system of the used Streptomyces, or as described, interacts with this, effecting secretion of cofactor-containing proteins in bacteria of the genus Streptomyces.

In a separate embodiment, the microorganism is characterized in that the folding of the amino acid sequence coded by the nucleic acid sequence a) occurs in the cytoplasm of the microorganism. This is of considerable importance, as many proteins having a cofactor are already partially or completely folded in the cytoplasm, wherein they are then capable of taking up the cofactor generally present in the cytoplasm of the cell. In order to be able to take up a cofactor, the tertiary structure of the protein must therefore be at least partially or completely formed. Secretion of such a protein that has already at least partially assumed its tertiary structure is generally disproportionately more complicated in comparison to the ejection of an amino acid sequence in its primary structure or, at best, secondary structure. In the first named case it is necessary, at least as far as possible, to retain the tertiary structure, i.e., the spatial form—for example, also so as not to lose again a non-covalently bound cofactor—, whereas in the second case, a not yet folded protein is secreted, which first assumes its tertiary structure after the secretion step. The ejection of such cofactor-containing proteins whose tertiary structure has already formed in the cytoplasm, especially those heterologously expressed in the bacterium, therefore represents a particular challenge made possible with the present invention, principally in regard to biotechnological fermentation processes for recombinant production of such cofactor-containing proteins. Consequently, in a preferred embodiment the microorganism secretes at least the amino acid sequence coded by the nucleic acid sequence a) together with at least one cofactor.

Cofactors are classified into different groups. Two large groups are the coenzymes and the prosthetic groups. Coenzymes typically are not proteins but rather are organic molecules that often carry chemical groups or serve to transfer chemical groups between different proteins or subunits of a protein complex. They are generally non-covalently bonded with the protein, particularly the enzyme that carries them. As cofactors, inventively particularly preferred coenzymes are chosen from nicotinamide dinucleotide (NAD⁺), nicotinamide dinucleotide phosphate (NADP⁺), coenzyme A, tetrahydrofolic acid, quinones, especially menaquinone, ubiquinone, plastoquinones, vitamin K, ascorbic acid (vitamin C), coenzyme F420, riboflavin (vitamin B2), adenosine triphosphate S-adenosyl methionine, 3′-phosphoadenosine-5′-phosphosulfate, coenzyme Q, tetrahydrobiopterin, cytidine triphosphate, nucleotide sugar, glutathione, coenzyme M, coenzyme B, methanofuran, tetrahydromethanopterin, methoxatin. However, the invention is not limited to these coenzymes as cofactors; rather, all further coenzymes represent cofactors in the context of the invention.

Prosthetic groups form a permanent part of the protein structure and in general are covalently bound to the protein, especially the enzyme. As the cofactor, the prosthetic group is preferably chosen from flavin mononucleotide, flavin adenine dinucleotide (FAD), pyrroloquinoline quinone, pyridoxal phosphate, biotin, methylcobalamin, thiamine pyrophosphate, heme, molybdopterin and disulfides or thiols, especially lipoic acid. However, the invention is not limited to such prosthetic groups as cofactors; rather, all further prosthetic groups represent cofactors in the context of the invention.

In a further preferred embodiment of the invention, the cofactor of the protein for which nucleic acid sequence a) codes is a coenzyme or a prosthetic group. In particular, coenzymes or prosthetic groups of this type can be present in various oxidation states. Moreover, the cofactor can concern a coenzyme or a prosthetic group. However it is also possible that the cofactor includes a plurality of coenzymes or a plurality of prosthetic groups, especially two, three, four, five, six, seven or eight coenzymes or two, three, four, five, six, seven or eight prosthetic groups or combinations thereof. As cofactors are frequently important in electron transfer processes and, for example, are often components of enzymes that catalyze redox reactions, they can be present in different oxidation states. Thus NAD⁺, NADP⁺ or FAD can be the oxidized compounds, whereas NADH, NADPH as well as FADH₂ can be the reduced counterparts. Analogously, cofactors can be present in their protonated or deprotonated form as the acid or base respectively, or generally—in so far as they alternate between a plurality of forms—can be present in all possible forms, for example, with or without the chemical group transferred from the cofactor under consideration, such as a methyl group or a phosphate group, as a quinone or hydroquinone, or as a disulfide or dithiol.

Furthermore it is possible that the amino acid sequence coded by nucleic acid sequence a) contains a cofactor assigned to neither of the two previously mentioned groups of cofactors. It is important that the amino acid sequence coded by nucleic acid sequence a) have above all a cofactor, wherein it is generally required for the presence of the cofactor that the amino acid sequence has a tertiary structure (i.e., has attained a higher degree of folding when compared with the amino acid sequence in its primary or secondary structure, wherein primary structure refers to the linear sequence of the individual amino acids and secondary structure to the existence of the basic structural elements α-helix and β-pleated sheet in the otherwise essentially linear amino acid sequence). Formation of a spatial configuration of secondary structural elements towards one another is part of the formation of the tertiary structure in the context of the present application. Additional cofactors can also be metal ions (trace elements), for example. Preferably, such cofactors concern divalent or trivalent metal cations such as Cu²⁺, Fe³⁺, Co²⁺ or Zn²⁺. Metal ions, for example, can facilitate the addition of the substrate or coenzyme, or can participate directly as a component of the active center or of the prosthetic group in the catalytic process. In addition, these metal ions can effect stabilization of the three-dimensional structure of proteins, especially enzymes, and protect them from being denatured.

In a particularly preferred embodiment of the invention, the amino acid sequence coded by nucleic acid sequence b) is a signal sequence for the Tat secretion path. As previously mentioned, Tat-dependent secretion enables ejection of completely folded polypeptide chains. Consequently, this secretion path is particularly suited for secretion of proteins having a cofactor. Accordingly, in bacteria of the genus Streptomyces, it is inventively preferred to use the Tat secretion path for secretion of heterologously expressed proteins having a cofactor.

Expression of a gene is its translation into the gene product(s) coded from this gene (i.e., into a protein or into a plurality of proteins). In general, the gene expression includes the transcription, that is, synthesis of a ribonucleic acid (mRNA) on the basis of the DNA (deoxyribonucleic acid) sequence of the gene and its translation into the corresponding polypeptide chain. Expression of a gene leads to formation of the corresponding gene product that exhibits a physiological activity and/or effects and/or contributes to a higher-level physiological activity, to which a plurality of different gene products are involved. In the context of the present invention, the gene product (i.e., the corresponding protein) is further complemented by a cofactor.

In a further preferred embodiment of the invention, the amino acid sequence coded by nucleic acid sequence b) and the amino acid sequence coded by nucleic acid sequence a) are components of the same polypeptide chain. In this way, Tat mediated secretion of a cofactor-containing protein is effected, especially of an enzyme, in that the Tat signal sequence fraction of the polypeptide chain interacts with the Tat-dependent translocation system of the Streptomyces in such a way that the cofactor-containing protein of the translocation system is ejected out of the Streptomyces cell. The Tat signal sequence fraction of the polypeptide chain therefore directs the whole polypeptide chain to a component of the Tat-dependent translocation system, in that it directly or indirectly binds to this component, wherein the bond is probably non-covalent.

Nucleic acids that code for such polypeptides can be produced by known processes for modification of nucleic acids. Some are illustrated, for example, in pertinent handbooks such as that from Fritsch, Sambrook and Maniatis, “Molecular cloning: a laboratory manual”, Cold Spring Harbour Laboratory Press, New York (1989). The principle is producing a nucleic acid that includes in the same reading frame nucleic acid sequences a)—the coding sequence for the cofactor-containing protein—and b)—the coding sequence for the Tat signal sequence, wherein nucleic acid sequence b) is preferably located up stream (i.e., at the 5′-end of nucleic acid sequence a)). Consequently, the Tat signal sequence is preferably located in the resulting polypeptide at the N-terminus of the polypeptide. A spacer can be optionally located between nucleic acid sequences b) and a) (i.e., between Tat signal sequence (Tat signal peptide) and the cofactor-containing protein to be secreted). The spacer can be 1 to 50, 1 to 40, 1 to 30, 1 to 20, 1 to 10, 1 to 8, 7, 6, 5, 4, 3, 2, or 1 amino acid long. On the nucleic acid level, this means that a spacer sequence is located between nucleic acid sequences b) and a), and based on genetic code, the spacer is three times as many nucleotides long as amino acids comprised in the spacer.

In a further preferred embodiment of the invention, the microorganism is chosen from Streptomyces lividans, Streptomyces coelicolor, Streptomyces avermitilis, Streptomyces griseus, Streptomyces olivaceus, Streptomyces hygroscopicus, Streptomyces antibioticus, and Streptomyces clavuligerus. The microorganism is preferably Streptomyces lividans.

Such bacteria are characterized by short generation times and low demands on cultivation conditions. In this manner, cost effective processes can be established. Moreover, there exists an extensive wealth of experience with bacteria in fermentation technology. For a wide variety of reasons that have to be experimentally determined for each individual case, such as nutrient sources, product formation rate, time required etc., various bacterial strains can be suitable for a specific production.

Gram-positive bacteria of the genus Streptomyces differ from gram-negative bacteria in that they immediately release secreted proteins into the medium surrounding the bacteria, generally the culture medium from which, when desired, the expressed proteins can be directly recovered or purified. They can be isolated directly from the medium or be processed further. Therefore a secretion preferably occurs into the surrounding medium. In addition, gram-positive bacteria are related or identical to most of the organisms of origin of industrially important enzymes and themselves mostly produce comparable enzymes, so that they have similar codon usage and their protein synthesis apparatus is naturally appropriately configured.

Codon usage refers to the translation of the genetic code in amino acids (i.e., which nucleotide order (triplet or base triplet) codes for which amino acid or for which function, for example, the beginning and end of the area to be translated, binding sites for different proteins, etc.). Thus each organism, especially each production strain, possesses a defined codon usage. Bottlenecks can occur in the protein biosynthesis if the codons laying on the transgenetic nucleic acid in the host cell face a comparatively low number of charged tRNAs. In contrast, synonym codons code for the same amino acid and can be better translated depending on the relevant host. This optionally necessary transcription therefore depends on the choice of expression system. Especially for nucleic acid sequences expressed from unknown, possibly non-cultivatable organisms, an appropriate matching of codon usage can be necessary on the microorganism that is to express them.

Fundamentally, the present invention is applicable to all microorganisms of the genus Streptomyces, particularly all fermentable microorganisms of this genus, and leads to an increased production yield in fermentation that can be achieved by adding such microorganisms as the production organisms. Products formed during fermentation are proteins having a cofactor, especially enzymes, including enzymes that catalyze redox reactions. Examples include oxidases, peroxidases, hydrogenases, dehydrogenases, reductases, biotin-dependent redox enzymes, and CO₂-fixing enzymes.

In vivo synthesis of such product (i.e., by living cells) requires transfer of the associated gene into a microorganism according to the invention, that is, its transformation. Those microorganisms are preferred which can be genetically handled with ease, for example, in relation to transformation with the expression factor and its stable establishment. In addition, preferred microorganisms are characterized by good microbiological and biotechnological handleability. For example, this relates to ease of cultivation, high growth rates, low demands on fermentation media and good production rates and secretion rates for foreign proteins. Frequently, the optimum expression system for the individual case must be experimentally determined from the abundance of different systems available from the prior art. Those microorganisms, which can be regulated in their activity due to genetic regulation elements that are, for example, made available to the expression vector, but which can also be already present in these cells, represent preferred embodiments. For example, they can be stimulated to expression by controlled addition of chemical compounds that serve as activators, by changing cultivation conditions, or by attaining a specific cell density. This allows for very economical production of the products of interest.

The microorganisms can be further modified in regard to their demands on the conditions of culture, exhibit other or additional selection markers or express other or additional proteins. In particular, the microorganisms can concern those that express a plurality of products, especially a plurality of cofactor-comprising proteins, especially enzymes, and secrete them into the medium surrounding the microorganisms.

Microorganisms according to the invention are cultivated and fermented conventionally, for example, in discontinuous or continuous systems. In discontinuous systems, a suitable nutrient medium is inoculated with the microorganisms (host cells) and the product is harvested from the medium after an experimentally determined time. Continuous fermentations are characterized by the attainment of a flow equilibrium, in which, for a comparatively long time, cells partially die off but also grow again, with product removed from the medium.

The present invention is therefore suitable for producing recombinant proteins, especially enzymes. According to the invention this is understood to include all genetic engineering or microbiological processes that are based on incorporating genes for the products of interest into an inventive microorganism. In the context of the present invention, a gene of this type includes the nucleic acid sequences b) and a) that were previously mentioned in detail and which effect a secretion of the cofactor-containing protein coded by the nucleic acid sequence a), generally together with the Tat signal sequence (Tat signal peptide) coded by the nucleic acid sequence b), and it particularly preferably further includes one or more sequences, especially promoter sequences, for the expression of the nucleic acid sequences a) and b). In this regard the gene in question is inserted by means of vectors, especially expression vectors, but also by those that cause the gene of interest in the host organism to be incorporated into an already present genetic element such as the chromosome or other vectors. The functional unit of gene and promoter and possibly additional genetic elements is inventively designated as the expression cassette. However, for this it must not also necessarily be present as a physical unit.

In the context of the present invention, vectors refer to elements consisting of nucleic acids, which comprise a gene in the context of the present invention. They are able to establish the gene as a stable genetic element in a species or a cell line over several generations or cell divisions. Vectors, particularly when used in bacteria, especially plasmids, are therefore circular genetic elements. In gene technology, a differentiation is made between those vectors that serve the storage and thereby to a certain extent also the technical genetic work, the so called cloning vectors, and those that fulfill the function of realizing the gene of interest in the host cells (i.e., to enable the expression of the protein in question). These vectors are called expression vectors.

In the context of the present invention, the nucleic acid (the gene) is suitably cloned into a vector. Accordingly, a further inventive subject matter is a vector that in the context of the present invention comprises a gene. For example, this includes those vectors that derive from bacterial plasmids, from viruses or from bacteriophages, or essentially synthetic vectors or plasmids with elements from the most different origin. Vectors with each of the additional available genetic elements are able to establish themselves in the relevant host cells for several generations to as far as stable units. Accordingly, in the context of the invention, it is irrelevant whether they establish themselves extrachromosomally as their own units or are integrated into a chromosome or in chromosomal DNA. Whichever of the numerous systems known from the prior art is selected, depends on the individual case. The achievable number of copies, the available selection systems, principally among them resistance to antibiotics, or the ability to cultivate host cells that can take up the vectors, for example, can be decisive.

Expression vectors include partial sequences that enable them to replicate inventive microorganisms optimized for production of proteins and bring the comprised gene to expression there. Preferred embodiments are expression vectors that themselves carry the genetic elements required for expression. The expression is influenced, for example, by promoters that regulate transcription of the gene. Thus, the expression can occur by the natural, original, localized promoter with this gene, but also after gene technical fusion, both by a prepared promoter of the host cell on the expression vector and also by a modified or a completely other promoter of another organism or of another host cell. Expression vectors can be regulated by changing the conditions of culture or by adding certain compounds such as the cell density or specific factors. Expression vectors permit the associated protein to be produced heterologously (i.e., in a different cell or host cell as that from which it can be obtained naturally). In this regard, the cells can belong to quite different organisms or derive from different organisms. A homologous protein production from a host cell that naturally expresses the gene over an appropriate vector also lies within the field of protection of the present invention, in so far as the host cell is an inventively designed microorganism. This can have the advantage that natural, modification reactions in a context of the translation on the resulting protein can be carried out in exactly the same way as they would normally be in nature.

Moreover, additional genes can be included for a useful expression system, for example, those made available on other vectors and which influence inventive production of the protein having a cofactor and coded by nucleic acid sequence a). They can be modified gene products or those intended to be purified together with the inventively secreted protein, for example, to influence its enzymatic function. They can, for example, be other proteins or enzymes, inhibitors or such elements that influence the interaction with various substrates.

A further subject matter of the invention is represented by a process for preparation of a protein having a cofactor by means of a microorganism belonging to the genus Streptomyces, the process comprising the following process steps:
a) inserting a nucleic acid sequence that is not naturally present in the microorganism and containing at least the following sequence segments:
   i) nucleic acid sequence coding for a protein having a cofactor, and
   ii) nucleic acid sequence that is at least 20% identical to the sequence stated in SEQ ID NO.1 or is at least 20% identical to the sequence stated in SEQ ID NO.3 or is a structurally homologous nucleic acid sequence to at least one of these sequences,
   into a microorganism, wherein sequence segments i) and ii) are functionally coupled, and
b) expressing the nucleic acid sequence according to a) in the microorganism.

With this type of process it is therefore possible to produce cofactor-containing proteins with bacteria of the genus Streptomyces, especially in a biotechnological fermentation. Due to Tat-mediated secretion of a cofactor-containing protein, especially an enzyme, its purification or further processing in such a process is significantly easier. Furthermore, a process of this type particularly enables a satisfactory product yield in fermentation. All the previously mentioned aspects for the microorganisms and vectors according to the invention also apply to the process according to the invention, so that they will not be repeated again here, but reference is made to the previous embodiments.

Consequently, in a preferred embodiment, the process is characterized in that at least the amino acid sequence coded by nucleic acid sequence a) is secreted together with at least one cofactor from the microorganism.

In a further preferred embodiment the process is therefore further characterized in that the cofactor of the protein for which nucleic acid sequence a) codes is a coenzyme or a prosthetic group.

A microorganism according to the invention is preferably employed in the process according to the invention. Therefore, a further subject matter of the invention is represented by processes for the preparation of a protein having a cofactor, wherein the processes include as a process step the cultivation of a microorganism according to the invention as previously described that secretes the protein into the medium surrounding the microorganism.

Cofactor-containing proteins, especially enzymes, manufactured in this type of process find a wide variety of uses. These include in particular oxidases, peroxidases, hydrogenases, dehydrogenases, reductases, biotin-dependent enzymes, especially CO₂-fixing enzymes, or redox enzymes in general. For example, redox enzymes are employed for the enzymatic bleach in washing and cleaning agents. They are particularly used in the textile and leather industry for downstream processing of natural raw materials. Moreover, all enzymes manufactured according to the process of the invention can be employed as catalysts for chemical reactions, once again in the context of biotransformation.

Consequently, in a further embodiment of the invention the process is characterized in that the protein is an enzyme, especially one chosen from redox-enzyme, oxidase, peroxidase, hydrogenase, dehydrogenase, reductase, biotin-dependent enzyme, CO₂-fixing enzyme, protease, amylase, cellulase, lipase, hemicellulase, pectinase, mannanase or combinations thereof.

Proteins, particularly enzymes, are optimized and especially genetically modified for their proposed field of application so as to provide them with improved properties for their respective purpose. Enzymes produced in processes according to the invention can therefore be the respective wild type enzymes or further developed variants. Wild type enzymes refer to enzymes present in a naturally occurring organism or in a natural habitat which can be isolated therefrom. An enzyme variant refers to enzymes produced from a precursor enzyme (e.g., a wild type enzyme) by modification of the amino acid sequence. The amino acid sequence is preferably modified by mutation, wherein amino acid substitutions, deletions, insertions or combinations thereof can be undertaken. Incorporation of such mutations into proteins is known from the prior art and has long been known to the person skilled in the art of enzyme technology.

Fermentation processes per se are well known from the prior art and represent the actual industrial production step, generally followed by a suitable purification method for the produced product, for example, the recombinant protein. All fermentation processes suitable for producing recombinant proteins therefore represent preferred embodiments of this inventive subject matter. A process of this kind is considered to be suitable if an appropriate product is formed. Products formed during fermentation are considered proteins having a cofactor, including enzymes, among which are enzymes that catalyze redox reactions. Exemplary redox enzymes are inter alia oxidases, peroxidases, hydrogenases, dehydrogenases, reductases, biotin-dependent redox enzymes, CO₂-fixing enzymes.

Optimal conditions for the production processes employed as well as the microorganisms and/or products being produced have to be experimentally determined by the person skilled in the art with the help of previously optimized culture conditions of the strains in question, for example, in regard to fermentation volumes, medium composition, oxygen demand or stirring rate.

Fermentation processes wherein the fermentation is carried out with a supply strategy can also be considered. For this the ingredients of the medium used up by the ongoing cultivation are fed in; this is also known as a feed strategy. Considerable increases in both the cell density and in the dry biomass and/or above all in the activity for the product of interest can be achieved in this way.

Analogously, fermentation can also be designed so that unwanted metabolic products can be filtered off or neutralized by addition of buffer or matching counter ions.

The manufactured product can be subsequently harvested from the fermentation medium. It is preferably inventively secreted into the medium. This fermentation process is correspondingly preferred over the product purification from the dry mass, but requires the availability of suitable secretion markers and transport systems.

Numerous combination possibilities for the process steps are conceivable for each product that is to be produced or is produced with microorganisms or processes according to the invention. The optimum process has to be determined experimentally for each particular case.

Microorganisms according to the invention are therefore advantageously employed in the described processes according to the invention and are used in these processes to produce a product, especially a protein that comprises a cofactor. Consequently, a further subject matter of the invention is therefore the use of an above-described microorganism for production of a protein having a cofactor.

In a preferred embodiment, the use is characterized in that the protein is an enzyme. The enzyme is advantageously chosen from redox-enzyme, oxidase, peroxidase, hydrogenase, dehydrogenase, reductase, biotin-dependent enzyme, CO₂-fixing enzyme, protease, amylase, cellulase, lipase, hemicellulase, pectinase, mannanase or combinations thereof.

The following example further exemplifies the present invention without limiting it in any way.

### EXAMPLE 1

Production of the cytosolic, FAD-containing enzyme choline oxidase from Arthrobacter nicotianae by Tat-dependent secretion in Streptomyces lividans—

All molecular-biological procedures were carried out by standard methods, as can be found, for example, in the manual by Fritsch, Sambrook and Maniatis, “Molecular cloning: a laboratory manual”, Cold Spring Harbour Laboratory Press, New York (1989), or in comparable specialized works. Enzymes, construction kits and equipment were employed in accordance with the respective manufacturer's instructions.

a) Construction of the Choline Oxidase Expression Vector—

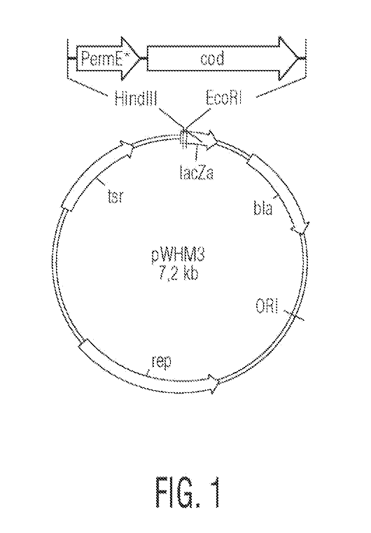

Based on the Escherichia coli-Streptomyces-Shuttle vector pWHM3 (Vara et al., J. Bacteriol., Vol. 171, pp. 5782-81 (1989)) a choline oxidase expression vector was constructed wherein a construct obtained by fusion-polymerase chain reaction (PCR) consisting of the strong constitutive promoter P_{ermE}* (Quiros et al., Mol. Microbiol., Vol. 28, pp. 1177-85 (1998)) and the gene of the choline oxidase from Arthrobacter nicotianae (cod, as listed in WO 2004/058955) were cloned into the segments Hindi II and EcoRI (see FIG. 1). The resulting plasmid for the cytosolic expression of the heterologous choline oxidase in Streptomyces lividans was called pKF1.

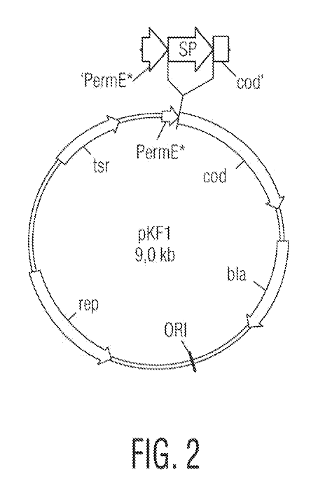

In the next step a Tat-specific signal peptide was introduced in order to enable the export of the protein together with its cofactor over the Tat path of Streptomyces lividans. For this, two different signal peptides from the closely related organism Streptomyces coelicolor were selected. The first concerns the signal peptide of the gene SCO00624 (putative secreted protein) and the other concerns the signal peptide of the gene SCO6272 (putative secreted FAD-binding protein) (Li et al., Microbiology, Vol. 151, pp. 2189-98 (2005)). Both constructs were obtained, in that a synthetic DNA fragment, which carries the DNA sequence flanked from corresponding regions to the plasmid pKF1, was employed as the megaprimer in an insertion mutagenesis (Method: Geisser et al., BioTechniques, Vol. 31, pp. 88-92 (2001)) (see FIG. 2). The QuikChange XL Kit from Stratagene (Stratagene/Agilent Technologies, Inc., Life Sciences and Chemical Analysis Group, Santa Clara, Calif., USA) was used for this.

The resulting plasmids for secretory production of the heterologous choline oxidase Streptomyces lividans were called pVR19 and pVR22 (Signal peptide SCO00624→pVR19, Signal peptide SCO6272→pVR22).

b) Secretion of the Choline Oxidase—

Expression and secretion of the choline oxidase was tested by transforming Streptomyces lividans TK23 (Kieser et al., (2005) Practical Streptomyces Genetics, John Innes Foundation, England) with the cod expression vectors pKF1, pVR19 and pVR22. Each cultivation was carried out in 40 mL rich medium consisting of:

| | | |
|---|---|---|
| Saccharose | 103 | g/L |
| Tryptic Soy Broth | 20 | g/L |
| MgCI₂•6 H₂O | 10.12 | g/L |
| Yeast extract | 5 | g/L |
| CaCI₂•2 H₂O (7.36%) | 20 | mL/L |

(pH 7.0-7.2)

In order to reduce formation of mycelium spheres, 500 mL baffled shake flasks containing an inserted metal spiral were incubated at 28° C. and 170 rpm. Samples were taken after 48 hour, 72 hour and 96 hour, centrifuged at 13,000 rpm for 3 minutes, and the supernatants were removed for the activity test.

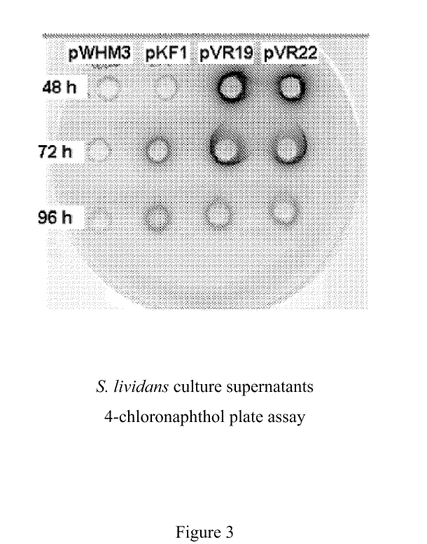

Activity of the choline oxidase was detected by using agar plates with 4-chloronaphthol (4-CN) which, on turning blue, indicate formation of hydrogen peroxide (S. Delgrave et al., “Application of a very high-throughput digital imaging screen to evolve the enzyme galactose oxidase”, Protein Engineering, Vol. 14, pp. 261-267 (2001)). With this method, the more hydrogen peroxide formed, the sooner a blue coloration of the medium appears. 50 μl of each supernatant were deposited in previously stamped holes on the 4-CN color plate. The result of the experiment is shown in FIG. 3. Supernatants of the 48 hour samples of the pVR19 and pVR22 strains already showed coloration after 1.5 h incubation, whereas the controls (empty vector pWHM3 and vector for the cytosolic expression pKF1) were negative. Extracellular activity of pVR19 and pVR22 decreased with increasing cultivation time. In the case of cytosolic expression, pKF1, a weak activity was also detectable after 72 h which is attributed to cell lysis.

This clearly demonstrates that microorganisms according to the invention are capable of efficiently secreting functional cofactor-containing proteins, particularly those normally localized in the cytosol.

We claim:

1. Microorganism comprising a nucleic acid sequence not naturally present in it, wherein the sequence comprises:
a) nucleic acid sequence coding for a protein having a cofactor, and
b) nucleic acid sequence that is at least 20% identical to the sequence stated in SEQ ID NO.1 or is at least 20% identical to the sequence stated in SEQ ID NO.3 or is a structurally homologous nucleic acid sequence to at least one of these sequences,
wherein the amino acid sequence coded by nucleic acid sequence b) functionally interacts with the amino acid sequence coded by nucleic acid sequence a) in such a way that at least the amino acid sequence coded by nucleic acid sequence a) is secreted from the microorganism, with the proviso that the microorganism belongs to the genus Streptomyces.

2. Microorganism according to claim 1 wherein the folding of the amino acid sequence coded by nucleic acid sequence a) occurs in the cytoplasm of the microorganism.

3. Microorganism according to claim 1 wherein it secretes at least the amino acid sequence coded by nucleic acid sequence a) together with at least one cofactor.

4. Microorganism according to claim 1 wherein the cofactor of the protein which nucleic acid sequence a) codes is a coenzyme or a prosthetic group.

5. Microorganism according to claim 1 wherein the amino acid sequence coded by nucleic acid sequence b) is a signal sequence for Tat secretion path.

6. Microorganism according to claim 1 wherein the amino acid sequence coded by nucleic acid sequence b) and the amino acid sequence coded by nucleic acid sequence a) are components of the same polypeptide chain.

7. Microorganism according to claim 1 wherein the microorganism is chosen from Streptomyces lividans, Streptomyces coelicolor, Streptomyces avermitilis, Streptomyces griseus, Streptomyces olivaceus, Streptomyces hygroscopicus, Streptomyces antibioticus, or Streptomyces clavuligerus.

8. Process for preparation of a protein having a cofactor by a microorganism belonging to the genus Streptomyces, the process steps comprising:
a) inserting a nucleic acid sequence not naturally present in the microorganism, the nucleic acid sequence comprising the following sequence segments—
i) nucleic acid sequence coding for a protein having a cofactor, and
ii) nucleic acid sequence that is at least 20% identical to the sequence stated in SEQ ID NO.1, or is at least 20% identical to the sequence stated in SEQ ID NO.3, or is a structurally homologous nucleic acid sequence to at least one of these sequences,
into a microorganism, wherein sequence segments i) and ii) are functionally coupled,
b) expressing the nucleic acid sequence according to a) in the microorganism.

9. Process according to claim 8 wherein the amino acid sequence coded by nucleic acid sequence a) is secreted from the microorganism together with at least one cofactor.

10. Process according to claim 8 wherein the cofactor of the protein which the nucleic acid sequence a) codes is a coenzyme or a prosthetic group.

11. Process for preparation of a protein comprising a cofactor comprising the process step of cultivating a microorganism according to claim 1, wherein the microorganism secretes the protein into the medium surrounding the microorganism.

12. Process according to claim 8 wherein the protein is an enzyme chosen from redox-enzyme, oxidase, peroxidase, hydrogenase, dehydrogenase, reductase, biotin-dependent enzyme, CO₂-fixing enzyme, protease, amylase, cellulase, lipase, hemicellulase, pectinase, mannanase or combinations thereof.


SEQUENCE LISTING (4 sequences)

SEQ ID NO: 1 - DNA, 102 bp, Artificial
  Tat signal sequence functional in Streptomyces
atgcacgagccgcacctcgaccgccgtctgttcctgaagggcacggccgtcaccggcgccgccctcgcactgggcgccaccgccgcgcccaccgcctccgcc

SEQ ID NO: 2 - protein, 34 aa, Artificial
  Synthetic Construct
MHEPHLDRRLFLKGTAVTGAALALGATAAPTASA

SEQ ID NO: 3 - DNA, 141 bp, Artificial
  Tat signal sequence functional in Streptomyces
atgaccgaggtgtcccgccggaaactcatgaaaggcgccgcggtgtccggcggcgcgctggcgctgcccgcgctcggcgcaccgccggcgacggccgcgcccgccgcaggccccgaggacctccccggccccgcagcggcc

SEQ ID NO: 4 - protein, 47 aa, Artificial
  Synthetic Construct
MTEVSRRKLMKGAAVSGGALALPALGAPPATAAPAAGPEDLPGPAAA